(19) United States
(12) Patent Application Publication
(10) Pub. No.: US 2026/0256385 A1
(43) Pub. Date: Sep. 3, 2026

(54) System and Method for Generating an Audiogram and Application Thereof to Personalise Frequency Response in a User Device

(71) Applicant: Bergen Audio Innovation, Bergen (NO)

(72) Inventors: Nadine Kroher, Bergen (NO); Aggelos Pikrakis, Bergen (NO); Petros Giannakopoulos, Bergen (NO)

(21) Appl. No.: 19/322,394

(22) Filed: Sep. 8, 2025

(30) Foreign Application Priority Data
Feb. 28, 2025 (GB) 2502922.4

(51) Int. Cl. A61B 5/12; A61B 5/00; H04R 3/04

(52) U.S. Cl. CPC A61B 5/125; A61B 5/7264; H04R 3/04

(57) ABSTRACT

An audiometry system, is based around a neural network trained using a reinforcement learning regime. Training of the system is based on historical audiograms and their relationships to known audiograms for test subjects having known characteristics, including known demographics, physical traits and noise exposure histories and occupation. According to its trained policy, the system presents a first state pairing at a first test frequency and a first selected sound intensity level, and then moves to a second selected different state pairing regardless of an interactive response determined by the system. With each presented state pairing, the system awards a negative reward, and also records a response to presented audio stimuli. The second state pairing is selected based on a probability outcome from the neural network taking into account the current state of the environment, sₜ, together with other parameters.

### CROSS-REFERENCE TO RELATED APPLICATIONS

This application claims priority to GB Patent Application 2502922.4 by Kroher, et al., titled: “SYSTEM AND METHOD FOR GENERATING AN AUDIOGRAM AND APPLICATION THEREOF TO PERSONALISE FREQUENCY RESPONSE IN A USER DEVICE”, filed on Feb. 28, 2025, which is incorporated herein by reference in its entirety for all purposes.

### FIELD OF TECHNOLOGY

This patent application relates generally to audiogram generation, and more specifically to audible frequency adaptation.

### BACKGROUND

This invention relates, in general, to a system and method generating an audiogram and is particularly, but not exclusively, related to the application of neural network technologies to assess rapidly the ability of individuals to perceive audible frequencies relative to nominally perfect hearing. The invention relates, also, to a system in which generated audiograms are applied to headphones (and the like), especially to personalise the frequency response thereof to compensate for imperfect hearing.

### SUMMARY

According to a first aspect of the invention there is an audiometry system based on a neural network trained using a deep learning reinforcement process set up with an agent and a policy based on temporal difference learning, the audiometry system arranged to generate an audiogram in which a hearing threshold at a plurality of selectable test frequencies is identified, wherein the system is configured to: generate frequency-level pairings on a selective basis dictated by the neural network, each frequency-level pair representing a current state of the environment; record, in memory, perception responses to those selectively generated frequency-level pairings presented from audio output device; and identify a hearing threshold for those selectively generated frequency-level pairings to assemble the audiogram from perception responses for a plurality of different frequency-level pairings; wherein selection of each frequency-level pairing takes into account the current state of the environment, sₜ, together with previous perception responses for different frequency-level pairings and defined parameter data pertaining to personal characteristics.

In an embodiment, the system is configured such that one of: the agent, as part of its action space, is arranged to select a next frequency to be assessed; selection of the next frequency follows a predetermined sequence independent of the agent; and in both configurations the agent is arranged to determine the level.

In an embodiment, the audiometry system is configured such that selection of frequency-level pairings can be subject to an applied mask configured to limit selection of test-frequency pairings. The mask predicts hearing thresholds at selectively omitted test frequencies.

In another aspect of the invention there is provided an audio output system including control circuitry configured to be dynamically adaptable to support adjustment of a frequency response of the audio output system through adaptation of processing parameters overseen by the control circuitry, wherein the control circuitry is responsive to data relating to hearing thresholds in frequency-level pairings determined through an on-line networked interaction of the audio output system with an RL-based audiometry system according to any of claims 1 to 10.

Modification of the frequency response of the audio output system, to compensate for determined hearing loss thresholds values over a range of frequencies, may be further modified to take into account manufacturer-specified design performance of the frequency response of the audio output system.

In a further aspect of the invention there is provided a hearing evaluation system based on a neural network trained using a deep learning reinforcement process to set an agent policy and in which evaluation system a system intelligence is arranged to control overall operation of the evaluation system, the neural network trained on a dataset of historical audiograms augmented by at least demographic data associated with each historical audiogram, wherein a system intelligence is configured to: record a negative reward for each presented state pairing of a frequency-sound intensity level; and following presentation of a first state pairing at a selected first test frequency and a selected first sound intensity level, to move to a second different state pairing regardless of an interactive response determined by the system intelligence; record, as received by the system intelligence, a first subject response with an association to the first state pairing; and select and present a second state pairing at a different second test frequency and different second sound intensity level, wherein selection of the second state pairing is based on a probability outcome for the neural network taking into account at least the first subject response; oscillate, at least once, between the first and second frequencies whilst respectively changing respective sound levels presented thereat, thereby to identify a threshold level of hearing at each of the first and second frequencies; and generate a visual representation of an audiogram based on reported positive responses to audio detection determined to be at a subject's threshold level of hearing at each frequency; and whereby consequently the agent policy is adapted to influence hearing loss evaluation strategy through selection of further state pairings based on responses to current and historical state environments and data related at least one of: (a) physiological characteristics of a test subject; (b) demographic data for a test subject; and (c) descriptive information describing a test subject.

In yet another aspect of the present invention there is provided a method of evaluating a hearing level threshold, the method comprising: training a neural network using a deep learning reinforcement process set up with an agent and a policy based on temporal difference learning, generating frequency-level pairings on a selective basis dictated by the neural network, each frequency-level pair representing a current state of the environment; recording, in memory, perception responses to those selectively generated frequency-level pairings presented from audio output device; and identifying a hearing threshold for those selectively generated frequency-level pairings to assemble the audiogram from a plurality of perception response at a plurality of different frequency-level pairings; wherein selection of each frequency-level pairing takes into account the current state of the environment, sₜ, together with previous perception responses for different frequency-level pairings and defined parameter data pertaining to personal characteristics, and the method further includes: generating an audiogram in which a hearing threshold at a plurality of selectable test frequencies is identified; and the system is configured to output the audiogram either on a user interface or transmitting data from the audiogram over a network to a device located remotely across the network.

In an embodiment of this method, amongst other things, the method may further comprise selecting and applying a mask configured to limit selection of test-frequency pairings. The mask predicts hearing thresholds at selectively omitted test frequencies.

In a further aspect, there is provide a method of modifying a frequency response of a speaker linked to a device and where the speaker has a manufacturer-defined frequency response, the method comprising: adapting processing parameters of the device, as overseen by control circuitry responsible for controlling soundwave generation from the speaker, in response to data identifying determined hearing thresholds at selected frequencies; and attenuating adaptation of the processing parameters when data identifying determined hearing thresholds at selected frequencies is determined to conflict with manufacturer-specified design performance of the frequency response of the speaker.

In yet a further aspect of the invention there is provided a method of estimating an audiogram, the method comprising: training a neural network using a deep learning reinforcement process to set an agent policy and in which the neural network is trained on a dataset of historical audiograms augmented by at least demographic data associated with each historical audiogram; recording a negative reward for each presented state pairing of a frequency-sound intensity level; following presentation of a first state pairing at a selected first test frequency and a selected first sound intensity level, selecting and moving to a second different state pair regardless of a determined interactive response; recording a first subject response with an association to the first state pair; and selecting and presenting a second state pair at a different second test frequency and different second sound intensity level, wherein selection of the second state pair is based on a probability outcome for the neural network taking into account at least the first subject response; oscillating, at least once, between the first and second frequencies whilst respectively changing respective sound levels presented thereat, thereby identifying a threshold level of hearing at each of the first and second frequencies; and generating a visual representation of the estimated audiogram based on reported positive responses to audio detection determined to be at a subject's threshold level of hearing at each frequency; and whereby consequently the agent policy is adapted to influence hearing loss evaluation strategy through selection of further state pairings based on responses to current and historical state environments and data related at least one of: (a) physiological characteristics of a test subject; (b) demographic data for a test subject; and (c) descriptive information describing a test subject.

These and other embodiments are described further below with reference to the figures.

### BRIEF DESCRIPTION OF THE DRAWINGS

The included drawings are for illustrative purposes and serve only to provide examples of possible structures and operations for the disclosed inventive systems, apparatus, methods, and computer program products for audiogram generation. These drawings in no way limit any changes in form and detail that may be made by one skilled in the art without departing from the spirit and scope of the disclosed implementations.

FIG. 1 illustrates a typical audiogram.

FIG. 2 shows a diagrammatic representation of an agent operating on a reinforcement learning regime, as contemplated in one or more embodiments of the present invention during training and subject assessment.

FIG. 3 is a representation of an RL-based ANN structured to provide acoustic loss evaluation according to one or more embodiments of the present invention.

FIG. 4 is a representation of a typical artificial neural network containing layers of neurons trainable and usable within the context of one or more embodiments.

FIG. 5 shows an overall system environment supporting the audiology system of one or more embodiments.

FIG. 6 shows an exemplary process by which a threshold of hearing determination is assessed for the exemplary case of two frequencies within an overall audiogram, performed in accordance with one or more embodiments.

FIG. 7 shows audiograms generated by a masked model approach in accordance with one or more embodiments.

### DETAILED DESCRIPTION

### Technological Background

As one of the five senses, hearing provides an important role in overall life experiences. Deterioration or indeed inherent impairment of this sense can impair quality of life, and almost certainly is at least annoying for both the sufferer and a third-party with whom the sufferer is interacting through conversation. Multiple factors can and do, in fact, degrade or influence a human's hearing range. These include age, short- and long-term exposure to loud/intense noises, upper respiratory (ear & nose) disease and genetic abnormalities. For instance, as people age, their ability to hear higher frequencies tends to diminish. One such study is discussed in the paper by Masterson, E. A., Tak, S., Themann, C. L., Wall, D. K., Groenewold, M. R., Deddens, J. A. and Calvert, G. M., 2013, “Prevalence of Hearing Loss in the United States by Industry.” American Journal of Industrial Medicine, 56 (6), pp. 670-681.

Inherent hearing impairment or gradual hearing loss is generally associated with the physical structures of, predominantly, the middle and inner ear, although neurological and brain functions also contribute to overall loss. Physiological degradation can arise from the impact of noise exposure on individuals from high noise environments in the workplace, whilst structural abnormalities may simply exist or develop over time. Some hearing loss can be mechanically compensated by external devices, i.e., hearing aids, whereas other more fundamental problems might require a more radical approach, such as with a cochlear implant. Both can make use of hearing test systems which evaluate hearing loss over the range of human hearing, typically being frequencies between about twenty Hertz (20 Hz) to about twenty thousand Hertz (20 kHz). The lower frequencies relate to the deep bass range that many more often feel rather than hear, such as the rumble of an earthquake or the lowest notes on a large pipe organ. Very high-pitched sounds, i.e., the high-end frequencies, correlate to things like the ringing of cymbals or the chirping of birds.

Hearing is a function of varying air pressures being converted into electrical signals which are then interpreted by the brain. The tympanic membrane, i.e., the eardrum, is oscillated by incident sound waves with the result being that force is transmitted through the ossicles (hammer, anvil, stirrup bones of the middle ear) that act as levers to the oval window. A large motion of the eardrum leads to a smaller but more forceful motion at the oval window. This pushes on the incompressible fluid of the inner ear and waves begin moving down the basilar membrane within the cochlea. The basilar membrane is an organ that functions to separate frequency components of sound, and then to propagate corresponding frequency-related stimuli to the brain via the auditory nerve. Different frequencies cause localised amplitude maxima in the wave as it travels down and along the basilar membrane. Relatively high frequencies get maximum response at the cochlear base [a connection of the membrane closest to the oval window], whereas lower frequencies are identified further into the basilar membrane towards its furled end and its apex. In the inner ear, the Organ of Corti is a spiral structure lying on the basilar membrane and functions to convert the membrane's motion into neural impulses. More particularly, hair cells in the Organ of Corti connect to a neuron that takes the signal to the central auditory system.

A related aspect to frequency response is dynamic range. Again, this is affected by hearing loss, and any improvement can accentuate listener pleasure (whether the sound is heard through a pair of headphones or a speaker system). A song that provides noticeable variations in level is almost always more engaging than one that stays pretty much the same from start to finish, so enhancing a user's hearing experiences by compensating for hearing loss can permit almost imperceptible differentiation (rather than possible silence) between musical constructs of a song, thereby making the listening to music pleasurable and compelling. Hearing noticeable variations in level is almost always more engaging than one that stays pretty much the same from start to finish, whilst (a) too wide a dynamic range impacts negatively on hearing the quiet parts clearly without the loud parts being uncomfortably loud and (b) a too small dynamic range means that the music will sound squashed and might even be fatiguing to one's ears, particularly when listened to at high volume/loudness levels.

Early identification of hearing loss is desirable (to say the least). The nature of and access to current testing regimes, however, does not necessarily support this objective. Hearing test systems evaluate loss by mapping a test subject's physical responses to the generation of selected discrete test frequencies [presented to the test subject] at varying volume, i.e., loudness, levels for each ear. This assessment is presented as an audiogram in which a relative loss of hearing, presented at quantised decibel (dB) levels for specific frequencies, shows the individual's hearing capabilities at different frequencies relative to someone with nominally perfect hearing. An audiogram shows a dB hearing loss “dB HL” offset relative to a zero dB requirement for a hearing threshold across the human hearing response range as typically assessed using about eight specific frequencies. The metric dB HL thus refers to the minimum sound pressure level a person can hear at a specific frequency relative to the level for a person with perfect hearing. With an audiogram, any positive dB HL value indicates a hearing loss, whereas a 0 dB assessment is considered normal and a reference audiometric zero datum for averaged hearing levels for normal young adults at each specific test frequency. dB HL is therefore an indication of the severity of hearing loss.

The different degrees of hearing loss have been generally qualified as:
None: Hearing within the normal range, with a threshold of 0-25 dB.
Mild: Difficulty hearing distant or whisper-level faint speech, with a threshold of 25 dB-40 dB.
Moderate: Difficulty hearing conversational speech, with a threshold of 41 dB-55 dB.
Moderately severe: Difficulty hearing most speech, with a threshold of 56 dB-70 dB.
Severe: Difficulty hearing loud speech and environmental sounds, with a threshold of 71 dB-90 dB.
Profound: Difficulty hearing even very loud sounds, with a threshold of >90 dB.

The dB scale describes the loudness of a sound, with a 3 dB change perceptible to the human ear. Since dB metrics are a logarithmic scale, 3 dB represents a doubling of the sound's intensity/power or, conversely, a halving in the power in the signal. For example, a sound that increases from 80 dB to 83 dB, depending upon the acuteness in hearing deficiency, might represent a perceptible increase generally around a given frequency.

With hearing loss associated with one or more of progressive degenerative and/or temporally-varying physiological conditions arising, for example, from age or a medical condition, if hearing loss assessment can be more readily accessed and assessed then compensational measures can be timely and more speedily taken to administer and, indeed, then regularly adjust a compensatory hearing-aid device. Each corrective hearing aid operates to expand its frequency response on a personalised basis. More particularly, armed with an accurate personal audiogram reflecting an individual's relative decibel hearing loss “dB HL”, an appropriate hearing aid can be built and administered, or control algorithms or physical mechanical structures in an existing hearing aid can be tweaked to optimise its frequency response. In this respect, the design or operation of a digital equaliser, amplifier and/or filter, within the output signal path of the hearing aid, can be produced selectively to boost loudness (or suppress) frequencies to improve clarity in, and an overall frequency range of, hearing.

Some modern digital hearing aid devices allow for customisable settings to be accessed through the hearing aid's app or remote control.

Traditional hearing tests are iterative in nature and were conducted by audiometrists or audiologists. These tests present frequency stimuli, namely specific tones presented at specific levels of sound intensity/power, to a test subject. Mechanical responses (such as the pressing or release of a button upon specific tone resolution) are then recorded to reflect hearing capabilities, with the test adjusting levels until the threshold from audible to not audible is found. Oscillating the level about the determined threshold for a specific frequency is used to confirm the hearing response of the individual under examination/test. The tests are conducted independently for each ear. During this procedure, skilled audiometrists would exploit acquired specialised and detailed empirical knowledge of likely hearing patterns over frequencies to influence the selection of stimuli to identify the hearing threshold. Age profiles were one such influencing consideration since increasing age has been observed statistically to affect hearing. This is somewhat just informed guesswork, but it does not infrequently rely on a brute force approach [where volume is steadily and successively increased at a frequency before a threshold is passed, whereafter the threshold is tested again by decreasing the volume to confirm the level at which hearing cognisance for the specific frequency is achieved/lost] because statistically findings are only indicative and not deterministic for an individual and their unique history and physiology. It thus makes use of a practitioner with varying levels of experience and thus subjective competence, and regardless it invariably requires a specialised environment, such as some form of anechoic chamber or the like to permit attention focus and to eliminate distracting extraneous sounds.

In any event, the existing hearing test processes are time consuming. The time aspect has commercial implications in the assessment or regular assessment of hearing loss/deficiency. Throughput of large numbers of people, particularly social or collaborative groups (e.g., aircraft engineers in a civil aviation or military role), can become difficult or somewhat prohibitive in the context of effective evaluation of an entire population or just a large workforce. Reducing the number of “trials” and consequently the time needed to perform a hearing test by honing the test towards a final personalised audiogram increases the scalability of the evaluation procedure (i.e., for mass testing), increases the throughput of hearing test facilities, increases the ability for decreased interval and thus regular testing, and lowers the barrier of subjects to agree to taking a test. Simplification of the test and/or related access to an online test application in a social environment rather than more of a clinical setting would also have beneficial impact.

Most modern hearing tests rely on the automated Hughson-Westlake procedure which does not require human intervention.

This Hughson-Westlake method, however, follows a predefined testing protocol which does neither take demographic factors into account, nor does it dynamically adapt to responses given throughout the procedure; it is essentially brute force. As a consequence, the process takes longer and, while saving on staffing requirements as a consequence of the automated approach, is more cumbersome for the test subject(s). This method simply ignores factors, such as demographics, that could be influential.

An alternative modern-day approach is “Audiogram Estimation via Gaussian Process Classification.” This is described by Barbour, D. L., Howard, R. T., Song, X. D., Metzger, N., Sukesan, K. A., DiLorenzo, J. C., Snyder, B. R., Chen, J. Y., Degen, E. A., Buchbinder, J. M. and Heisey, K. L., 2019. “Online Machine Learning Audiometry”, Ear and Hearing, vol. 40(4), pp. 918-926.

Before turning to the inventive approach of the present invention in using a neural network, it is important to recap on some of the basic attributes of artificial neural networks “ANNs.” Reference is briefly made to the following articles that provide some background to for a learning in neural networks:
1. Mnih, V., Kavukcuoglu, K., Silver, D., Rusu, A. A., Veness, J., Bellemare, M. G., Graves, A., Riedmiller, M., Fidjeland, A. K., Ostrovski, G. and Petersen, S., 2015. “Human-Level Control Through Deep Reinforcement Learning.” Nature, 518 (7540), pp. 529-533.
2. Watkins, C. J. and Dayan, P., 1992. “Q-learning” presented in Machine Learning, vol. 8, pp. 279-292.
3. Vaswani, A., Shazeer, N., Parmar, N., Uszkoreit, J., Jones, L., Gomez, A. N., Kaiser, Ł. and Polosukhin, I., 2017. “Attention Is All You Need.” Advances in Neural Information Processing Systems, vol. 30.

In essence, an ANN is merely a circuit of electrical components. It provides an approximation to reality and is thus an approximation machine. Whether implemented in hardware or software, an ANN consists of a collection of nodes (‘neurons’) arranged in layers and coupled to the neurons in the layer above and below by connections (‘links’). Each neuron receives inputs from neurons in the layer above and passes on its output to neurons in the layer below. Often each neuron in one layer will have connections from every neuron in the layer above, and to potentially every other neuron in the layer below, but this is not always the case. Each neuron has a ‘weight’ and a ‘bias,’ with the effect of these being that some neurons will feed inputs onto others strongly, whilst others may do so relatively weakly, or even negatively. An ANN is a network of intricately interconnected neurons which are equivalent to resistors and transistors and thus influence the network's function, whereas the links between neurons being equivalent to wire tracks on a PCB or the like. Consequently, in contrast with what is known to be a ‘computer,’ (see for example either of the well-accepted Harvard and von Neumann architecture definitions which both require a computer to possess inter alia an arithmetic logic unit “ALU” and interactional registers within a core CPU environment (e.g., the Harvard Architecture and/or the Von Neumann architecture), an ANN does not use a CPU and has neither a CPU nor an ALU within its nodal network.

The power of an ANN arises from its ability to provide a solution to an intractable problem that cannot be coded using a computer program. For example, a computer programmer would not be able to code a program for which the inputs were unknown and the resulting output uncertain because there is no evident path. Developing a network having an output representative with a desired outcome, i.e., the known “ground truth,” requires conceptual thought concerning the training of the ANN. This is not materially different in approach to defining the operational characteristics required by a finite impulse response filter, although the resulting ANN is much more complicated and the training process- and the definition of the objective test function-considerably more involved. Basically, the ANN is self-teaching in that an internal feedback loop allows its current suggested output to be tested against the known ground truth. Training is thus an initial iterative process by which the ANN's internal pathways are optimised by honing the weights and biases using test data over a succession of training epochs [in the sense of a supervised network] or episodes [in the case of a Reinforcement Learning “RL” network].

In a supervised network, a cost/loss function—an manifestation of conceptual learning objectives required to set up the ANN—may be assessed relatively simply in a vectorial comparison (whether in a regression scenario that makes use of a vector or number, or a classification scenario needing alignment to a correct “one-hot”) between the prediction and the desired response, i.e., the “ground truth.” The cost/loss function is used to quantify the error signal in the machine, with a backpropagation process applied to adapt neuron weights and biases in order to drive performance of the ANN towards its objective. Differently, in the RL paradigm, the assessment of the efficacy of the ANN is based on the quality of the policy of an agent within the context of a problem framed as a Markov Decision Process “MDP”, wherein the objective of the agent is to maximize received rewards in an agent-state-environment. In RL, the underlying premise is that intelligence of the network is an emergent property of the interactions between an “agent” and its environment, with intelligence being realized by the capacity of the agent to select the appropriate strategy to achieve the purpose/goals having regard to all possible behaviours, i.e., a teleologically-orientated approach. In MDP, the next action is dependent upon the preceding action, with MDP producing a set of nodes each representative of an available “state” condition. Each state thus aligns with an “action” [also termed an “anchor” or “edge”] within an environment. The environment is dependent upon the application in which the RL network is designed to function. In RL, the agent controls selection of the state in the environment with a view to maximize a calculated reward function having regard to alternative policy paths each seeking to achieve a defined end objective. The selection of each action is therefore based on a probabilistic approach. Each policy dictates the actions that the agent can take having regard to all possible available states in the environment, with selection of an optimised policy based on evaluation of alternative policies having regard to their respectively attributed overall reward functions. Policies therefore reflect a search for balance between exploration and exploitation of state-actions pairings. An RL approach thus can appreciate that the best overall strategy may require short-term sacrifices, meaning that the best approach may ultimately include some punishments or backtracking along the pathway of the policy.

By way of exemplary summary, a temporal error signal in RL can be considered to be analogous to a defined loss function [in a supervised learning approach]. Resolution of the temporal error signal is therefore associated with setting the policy of the agent and, ultimately, underpins the structure of a resulting ANN. More particularly, RL is a machine learning technique that trains a software emulation to make decisions to achieve the most optimal result. It mimics the trial-and-error learning process that humans use to achieve their goals. Actions that work [in the context of coding and evaluation during training] towards a goal are reinforced, whereas actions that detract from the goal are ignored or positively downgraded or “punished.” They learn from the feedback of each action and self-discover the best processing paths to achieve final outcomes aligned with a defined objective that, ultimately, is then applied/realised in the components of a finalised network. Expressing this differently, the agent interacts with its environment via actions for which rewards are received. RL is configured optimise a policy which details the actions that should be taken given a specific environment to maximise reward. The RL approach is capable of delayed gratification in which the reward/punishment is delayed for a number of training episodes. RL is an underlying intelligence that is applied to a real-world network to fix weighting factors of neurons that define synaptic pathways, with the definition of the state-action pairs being conceptual in nature and critically underpinning a derived solution to the technical objective.

In machine learning, once acceptable alignment to the ground truth is reached or the policy set, the values of the weights and biases of neurons in the network that realise these results are fixed. Digitalised sample data [representing a query] can then be cascaded, in a forward pass through the network, from the input to the output. Supervised learning and RL are machine learning approaches that both yield a final product, with the form of implementation in software or hardware being design alternatives.

As general background to the field of hearing loss determination as particularly determined by machine learning techniques, reference is made to the following papers:
a) “Online Machine Learning Audiometry” by the authors D. L. Barbour et al, published in Ear and hearing, 40 (4), pp. 918-926.
b) “Prevalence Of Hearing Loss In The United States By Industry” by the authors E. A. Masterson et al, published in American journal of industrial medicine, 56 (6), pp. 670-681.
c) “Human-Level Control Through Deep Reinforcement Learning” by the authors V.Mnih et al, published in Nature, 518 (7540), pp. 529-533.
d) “Q-Learning” by the authors C. J. Watkins et al, published in Machine learning, 8, pp. 279-292.
e) “Attention Is All You Need” by the authors A. Vaswani et al, published in Advances in neural information processing systems, 30.

### Overview

The embodiments of the present invention make use of a novel method for performing automated audiometry, based on Reinforcement Learning “RL.” Significantly lower number of trials are required to estimate an audiogram compared to prior art automated audiometry methods, such as 1) the online Hughson-Westlake procedure for audiogram estimation, and 2) the audiogram estimation via Gaussian Process Classification.

The present invention beneficially reduces the number of trials required in the evaluation of hearing loss, and consequently the time needed to perform a hearing test. This increases access to and scalability of hearing assessments and thus supports effective and efficient large-scale testing. Throughput at hearing test facilities is therefore increased, with this lowering the objections of—or barriers presented to—individuals to participate in hearing tests that can, upon identification of hearing loss, improve their quality of life through (a) provision of a suitable compensatory hearing-aid device, and/or (b) using the audiogram to optimise a speaker or headphone system's output performance, namely spectral components within its frequency response, towards the individual's specific and potentially changing audio sensory needs.

Observationally, the approach of the present invention has yielded accurate personal audiograms using approximately half the number of tones since the trained network is able to simulate, based on historical data utilised by the RL and subsequently “coded” into neurons, how a person responds to frequency-intensity pairings, i.e. state-action coupling. The resulting system thus supports a rapid evaluation approach supporting applications in clinical and mobile test environments, with the latter providing a statistically reasoned elimination of frequencies when approximation is more acceptable.

Each corrective system or output device/system of the invention thus operates—or can be designed—to expand the frequency response on a personalised basis to provide increased perception of tonal components across the spectrum to improve the listening/hearing experience. More particularly, armed with an accurate assessment of hearing loss expressed in terms of an individual's relative decibel hearing loss “dB HL” at test frequencies, control algorithms or physical mechanical structures can be employed and/or manipulated to optimise the frequency response to compensate for hearing imperfections that can be offset (partially or comprehensively) by a hearing aid or within the context of headphones (and the like). Compensation can be achieved, for example, through adaptation of, for example, a digital equaliser within the output signal path. More specifically, adaptation is designed to boost loudness at identified frequencies or bands of frequencies at which there are spectral component hearing deficiencies recorded in a personal audiogram.

The application of results from audiograms can, beneficially, also be used to reduce an overall sound level environment [otherwise potentially causing noise induced hearing loss affecting others] by supporting the dialling down or capping of output volumes generated by the sound system's amplifier and experienced through room-based speakers or, to a lesser extent, personal headphones. If overall volumes can be decreased whilst allowing the listener to perceive a fuller range of frequencies at lower levels, then the overall decrease in supplied local levels has an associated and corresponding reduction in sound leakage into the local environment in which non-engaged third-parties can reside. This is a consequence of the improved frequency perception and the elimination of the use of higher overall sound powers previously required by a user to compensate for any ability to resolve certain frequencies at lower dB levels. Expressing this differently, applying a personal audiogram to adapt, according to a user-determined schedule, speakers and headphones to a response equivalent to an audiometric zero result could be beneficial, especially when such compensation can be automatically applied following a user-friendly and simple (regularly or aperiodically taken) hearing test.

In the interactive environment in which, particularly, the output of headphones is responsive to digital processing that makes use of an ASIC and/or coding run by a digital signal processor “DSP” to oversee operational output, frequency response compensation at identified frequencies [related to identified hearing loss] can be controlled by how digital processing is applied to influence the delivered analog waveform. The principles of digital processing techniques to boost output are well-known.

### Technical Details

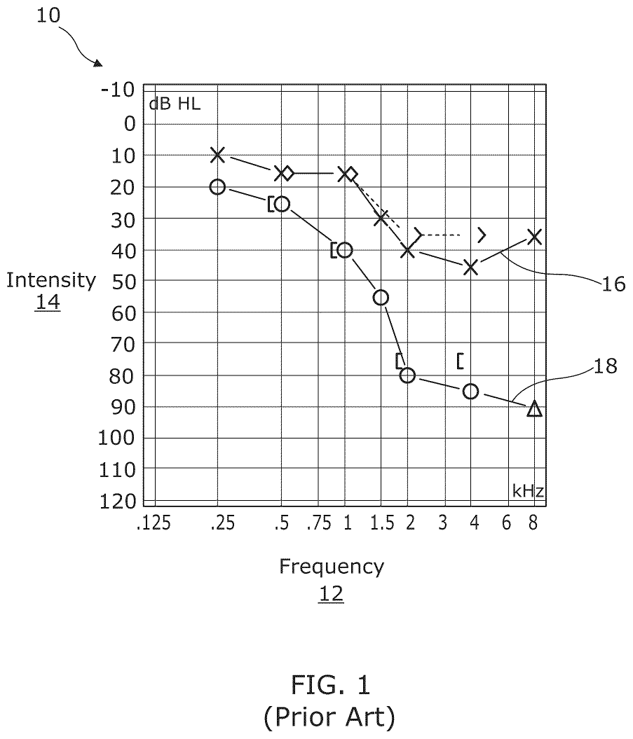

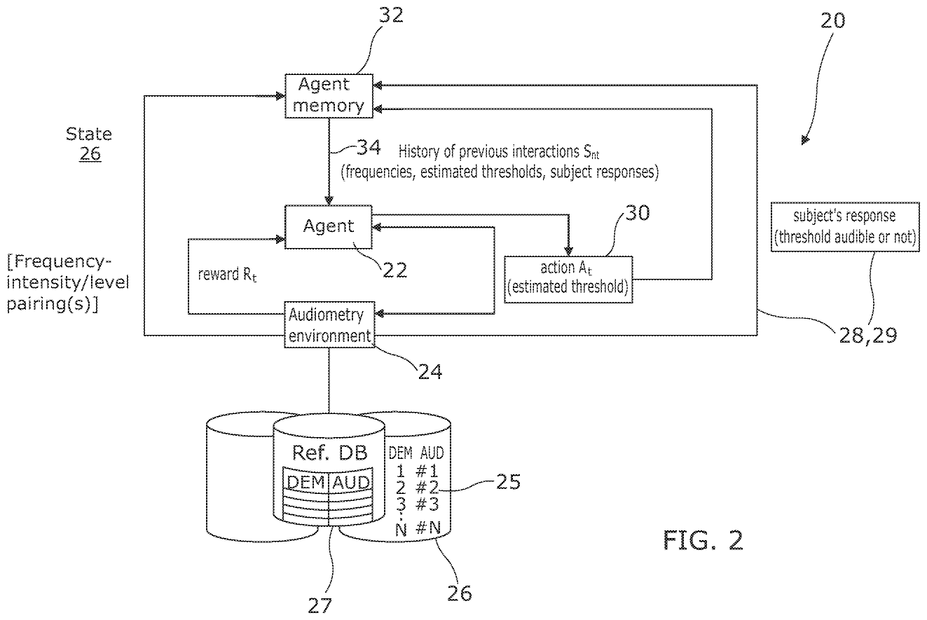

Referring briefly to FIG. 1, there is shown a typical audiogram produced following a standard procedure for assessing hearing loss. The abscissa 12 relates to varying- and indeed typical-test frequencies in kilohertz (kHz), whereas the ordinate axis 14 is sound intensity expressed in terms of relative loss in dB HL. The precise plots for, respectively, a test subjects audio capabilities for their left 16 and right 18 ears are exemplary and simply reflect that differences between hearing in each ear is possible/not uncommon, with this meaning that resolvable sound intensities relative to a baseline 0 dB HL [for nominally/accepted perfect hearing] can vary. For example, at 250 Hz, hearing in the left ear is compromised by a level of 10 dB HL (a positive value), whereas in the right ear hearing loss is 20 dB. At a higher frequency of 2 kHz, the respective and relative losses for this test subject increases to 40 dB HL and 80 dB HL, with this showing increased deafness in the right ear relative to the left. It is noted that a flat response at 0 dB HL represents hearing considered as “perfect.” Test frequencies, although not set in stone, can approximate to a logarithmic sequence, with a lower frequency typically around 125 Hz and an upper test frequency of around 6 kHz to 8 kHz. Other ranges are, of course, possible. For example, loss determination in the range of 15 kHz to 17 kHz may be warranted.

Usually, an audiogram will make use of eight frequencies distributed across the audible frequency spectrum, with dB HL variations at between 3 dB to about 10 dB steps and usually at incremental 5 dB steps. Of course, this is subject to design consideration for the test procedure, including any focus to identify deficiencies at particular frequency or frequency ranges using a sensibly resolvable dB HL granularity.

There are two aspects to the invention, namely (1) RL training, and (2) real-world evaluation of hearing loss of a specific individual using the trained ANN.

In overview, during the RL training regime, the embodiments of the present invention exploit identified hearing loss patterns and/or demographic data, including age and previous responses to investigation(s). This has the identified effect, once the network is trained and fixed appropriately by applying weighting factors that adequately generate the desired result, to reduce the time necessary to evaluate hearing loss in an individual.

During application of the trained ANN in hearing loss assessment/determination during the forward pass, the system functions to estimate a statistically more likely audiogram whilst limiting interactions. i.e., responses to audio tone at a frequency and volume, with a subject. This constitutes a trial as part of a longer-term strategy, with the goal of the trained ANN to arrive at a personalised audiogram without having to step through unnecessary test frequencies.

### A. Test Environments and Assessment Approaches

In terms of a test environment, during hearing loss assessment/determination, the tones can be generated within a specifically designed test chamber, or as an output from speakers or a headset worn by the subject. It is appreciated that speaker and headphone dynamic range and the ambient environment could compromise a final assessment of actual personal hearing loss, but that may be acceptable to enhance/change personal listening experiences through control of speaker and headphone operational parameters. In all cases, the trained system/ANN is designed to select the next frequency to be tested, and also an intensity level at which that selected frequency is to be presented.

There are two embodiments for the testing procedure in the forward pass, both of which are based on a network trained according to the underlying RL approach described herein.

An “exact” version finds specific application in clinical audiometry applications. The audiometry procedure is performed by an intelligent agent which decides the optimal next threshold value it should test at for a given frequency, based on the subject's past responses. This will be described in more detail below.

An “approximation” embodiment finds more ready application in, particularly but not exclusively, consumer applications where absolute deterministic precision in frequency perception can be waived for one of more reasons, including (a) test expediency, (b) the final environment in which sounds are heard (e.g., supplied through headphones worn on a temporary basis, such as when playing a video game and listening to any accompanying sound or soundtrack), and (c) test regime frequency in the sense that the consumer electronics can be updated more regularly with compensating factors. The approximation procedure is similar to the “exact” procedure, except the agent is not required to find the exact intensity threshold at each frequency but only at a subset of the frequency and missing values are inferred by a second machine learning model. In other words, by exploiting common hearing loss patterns, a second model can predict the threshold values at frequencies that have not been measured. Of course, there is no reason not to apply the exact approach to all applications, including consumer electronics.

This partial estimation of the audiogram [in the approximation embodiment] further reduces the number of trials required and is sufficient for non-clinical applications, such as more general adaptation of sound generating equipment. The further reduction in the number of trials (at frequencies of varying intensities) is possible by only using the agent to estimate the approximate thresholds for a subset of the frequencies, via interaction with the subject and inference of other responses across the entirety of the spectrum. A masked model, described below, is then used to predict the thresholds for the missing frequencies without requiring any further interaction with the subject. The mask further leverages the fact that, within the basilar membrane, physiological change and thus detection degradation at a local point has related implications for surrounding areas of the membrane. This means that frequency tests may be omitted, and results inferred from the application of an appropriately structured mask.

In a described embodiment, a consumer audiometry application utilizes the approximation version of the audiometry assessment scheme. This remote networked arrangement supports online audiometry testing by having the test subject interact with the assessment system via a web interface. Since the audiometry procedure is not performed in a controlled environment [with calibrated equipment], the embodiment is arranged to compensate for variables, including the frequency response of a user's headset, in order to generate an audiogram estimation with reasonable accuracy within an acceptable range. Again, more detail is provided below.

### B. Training of the Ann Based on Reinforcement Learning

To train the agent and thus to determine, ultimately, the fixed weighting factors applied to neurons within the agent (i.e., the ANN), a simulated audiometry environment is created in which the agent interacts with a dataset of historical recorded audiograms—this historical data are available from NIOSH [“Prevalence of Hearing Loss in the United States by Industry” referred infra]-referenced into specific individual subjects, their demographics (such as age and other relevant quantifiable parameters that can be leverage by the RL approach) and particularly responses to audio stimuli for each specific reference subject. The agent, according to the invention, interacts with an environment which can simulate a response, namely ‘yes’ or ‘no’ to hearing perception, at specific frequency-dB stimuli for a specific subject having specific hearing characteristics and personal attributes [recorded in an historical audiogram]. In each successive individual assessment, the agent takes an action in the form of selecting a dB-frequency stimulus in the expectation of successfully arriving at an identified dB HL threshold corresponding to the lowest discernible level presented on the historical audiogram for the specific subject. The agent is rewarded depending on how many trials are needed for its audiogram estimation to match the reference audiogram of the reference subject, with a higher reward associated with fewer trials needed to arrive at the actual threshold in the historical audiogram for the subject. In other words, there exists a known audiogram outcome, with the agent tasked to converge on this outcome in the shortest number of test steps. The agent, during training, operates to realise that the attributed value of an action is indicative of an accuracy of that action to the agent-controlled state/environment. The inventors have identified that the historical corpus of reference audiograms thus provides a map of how successive/subsequent test frequencies are related to former test frequencies [in terms of specific volumes and therefore dB HL-frequency pairing for a selected demographic characteristic]. The inventors have identified that this approach correlates to likely physiological degradation of parts of the basilar membrane.

Reference is now made to FIG. 2 which shows a diagrammatic representation 20 of an agent operating on a reinforcement learning regime, as envisioned by a preferred embodiment of the invention during training and subject assessment paths. There are numerous interactions between the principal components of the agent 22, the audiometry environment 24 and an agent memory 25.

In FIG. 2. the two time-separated operational paths are essentially identical albeit that (i) a first path relates to a simulated environment in which a reference subject's response is known from a pre-stored historical audiogram for the reference subject, and which response 28 is one of the reference subject's actual record of hearing capacity (or not, as the case may be) at a given frequency at a set dB level; and (ii) a second path 29 relating to an active assessment of a real individual generating an unknown response to the state [frequency-level] pairing selected as an investigating stimuli in a forward pass on a post-trained network. In both paths, the agent 22 selects the next frequency-level pairing either from the historical data (for training) or according to a test regiment (in a forward pass full assessment of actual hearing loss). FIG. 2 is thus applicable to both a stimulated environment and an actual test environment because the agent interacts with the prevailing environment in a similar fashion.

For both paths, an agent memory 32 stores the subject's response relative to the estimate of the action Aₜ 30 for the specific state 26. Each agent-generated action Aₜ is an estimated threshold of audio perception for the selected frequency-level pairing.

Again, for both paths, storing of a history of tests is fed 34 to the agent 22 for its use in determining the next frequency to be selected in re-generation of an historic audiogram [in the case of training] or an actual audiogram of an individual undergoing actual hearing loss assessment.

During training, the audiometry environment 24 is accessible reference data 25 stored in a database 26. The accessible reference data includes demographic data “DEM” cross-referenced to specific audiograms for specific reference subjects 1 to N, where N is a positive integer and the demographic data 27 (including age, sex and other selectable influencing factors, including age, sex, occupation, noise exposure, medical history (both prevailing (“I have a cold”) and historical, previous ear surgery, upper respiratory track infection(s), tinnitus, genetics, head trauma, ototoxicity, the presence of ear wax, etc. as will be readily understood to the skilled audiologist). The reference data 25 may further include a description of a profession or some other quality or qualifier for the subject, with this converted into a vector representation using Natural Language Processing “NLP.” Such qualities or qualifiers can include age, sex, occupation, noise exposure, medical history, previous ear surgery, upper respiratory track infection(s), tinnitus, genetics, head trauma, ototoxicity, the presence of ear wax, and/or ‘I was a drummer playing in a heavy metal band for a decade,’ or ‘I worked as a roadie assembling the soundstage’, etc. The descriptions provide context.

In the simulated training phase, each generated action Aₜ 28 for a selected frequency-level pair, i.e., a specific state, can be backwardly assessed with respect to the historical audiogram for the specific state, i.e., the specific frequency and its dB level under evaluation. This corresponds to a history of actions. The agent is arranged to generate a cumulative punishment related to each action that fails to produce coincidence with a ground-truth state in the historical audiogram. The more actions required to converge to the specific frequency and level in a particular/selected audiogram, the higher the relative debt for the suggested RL policy.

By way of an intermediate summary, the agent is attempting to set a policy that optimises identification of the hearing threshold using the fewest number of tests. Selection of the next frequency to be measured can either be part of the actions space of the agent, or otherwise it can follow a predetermined sequence independent of the agent (e.g., start with a test in the left ear at the lowest frequency, and then move to the next test frequency regardless of the response, moving only to the right ear after the highest frequency has been tested (regardless of threshold determination) in the left year. In both cases, the dB level is determined by the agent. The agent uses all of the information available in its memory. The state of the environment, sₜ, is the current frequency and the current level, with the response being a feedback signal sent back to the agent by the environment. The system is arranged also to augment the agent with a memory which stores all previous interactions with the environment. Each interaction in the agent's memory is a record of the frequency that was tested, the threshold estimated by the agent and the subject's response. The current state sₜ is added to the memory and, along with the history of previous interactions, forms a new state sₕₜ. which the agent uses to estimate a threshold Aₜ for sₜ. The threshold along with the subject's response are then also stored in the agent's memory. Subject to the implementation, the agent may receive from the environment the next frequency to measure, but it will always receive the level. The audiogram estimation process is preferably formulated as a Markov Decision Process (MDP), which is then optimised via reinforcement learning using a reward signal. The input is an enhanced representation of the state (frequency and intensity level) augmented by selected demographic data to produce Q(sᵢ,historyᵢ) or more particularly Q(fᵢ,levelᵢ,responseᵢ,ageᵢ,sexᵢ,parameterᵢ), with a resulting network thus trained on a history of available audiograms that make use of state-action pairs up to and including the current state, Q(s,a). In a preferred embodiment, the Q input is arithmetically encoded as a vectorial embedding.

Each individual audiogram within the historical dataset is, when successfully reproduced, treated as an episode. The RL agent, as applied in the present invention, is configured to identify patterns and to maximise rewards following pattern recognition.

To explain the approach of the preferred embodiment in more detail, the RL training regime makes general use of the Bellman equation for deep Q-Learning Q(s,a) where ‘s’ is the state and ‘a’ the action. This rewards the immediately following action from a previous state and the response. It is contemplated that a sparse reward scheme could be employed in which the final reward is determined at the end of each episode. As a general statement, attributed levels of reward are inversely proportional to the number of steps required by the policy to reach an optimum conclusion at the end of the episode. In each iteration, an update to the Q state action pair Q(s,a) [see eqn. (1) below) is supported by adding a quantity dependent upon both the reward and the next state as all is determined relative to the current state. This represents an applied approach in temporal difference learning.

$$Q ( s , a ) ← Q ( s , a ) + λ ( R ( s , a ) + γ \{ {Q}_{{max}_{{a}^{′}}} ( {s}^{′} , {a}^{′} ) \} - Q ( s , a ) ) \qquad eqn . ( 1 )$$

where:
Q(s,a) is the current Q-value for taking action, a, in the current state, s,
λ is the learning rate which determines how much emphasis is placed on the new information to override/adapt the older Q-value,
R(s,a) is the immediate reward received after taking action a in state s,
γ is the discount factor, which weighs future rewards relative to immediate rewards, and
Qₘₐₓ_{ₐ},(s′, a′) is the maximum Q-value for the next state, s′, across all possible actions a′.

This equation- or its equivalent for other network configurations and approaches-updates the Q-value based on the immediate reward and the maximum expected future reward. Through repeated application, the agent learns to approximate the optimal Q-values and can determine the best actions to maximize long-term rewards. This also means that, with the elapse of trials and time, the agent's ability to explore off-policy results is reduced, meaning that the agent's approach to adaptation of its applied policy becomes more conservative.

The preferred embodiment is configured to look at the final output layer of the neural network, with the neurons thereof providing a relative probabilistic evaluation at each of the output points. The approach of the present invention with respect to the setting of the weighting factors for neurons is to compute the A change between successive Q states, i.e., Q(s,a) relative to Q(s′,a′). The A change is used to drive updates in each neuron's weighting factors through known backpropagation techniques relative to the current output of the ANN (or more specially a QNN). A stable output of the network is achieved when there is acceptable convergence to the optimal Q(s,a) [where the network is stabilised, i.e., a solution to eqn,1].

In terms of the structure of the embedding, the RL process makes use of temporal evolution of the experimental history compared to static values, such as age and sex, etc. (see above). This produces a finite alphabet of test frequencies, say n=7, as suggested below in Table 1 albeit only containing an incomplete dB HL result for some of the frequencies, fᵢ:

TABLE 1

| | f₁ | f₂ | f₃ | . . . | fₙ |
|---|---|---|---|---|---|
| | dB₁ | dB₂ | dB₃ | . . . | dB₄ |
| response | Yes | No | No | . . . | Yes |
| => | 1 | 0 | 0 | . . . | 1 |

Making typical but preferred use of a one-hot matrix encoding, data manipulation and concatenated of the resulting vectors produces a numeric two-dimensional unfolded representation of fixed length for all available frequencies and all dB HL levels for each frequency, with a partial frequency matrix shown below:
Frequency
[10000000000000] [00 00 0 100 000 000] [0 0 1 0 0 0 0 0 0 0 0 0 0 0]- - - [0 0 0 0 0 0 0 0 0 0 0 0 0 1] producing a frequency vector [10000000000000,010000 . . . , etc.]

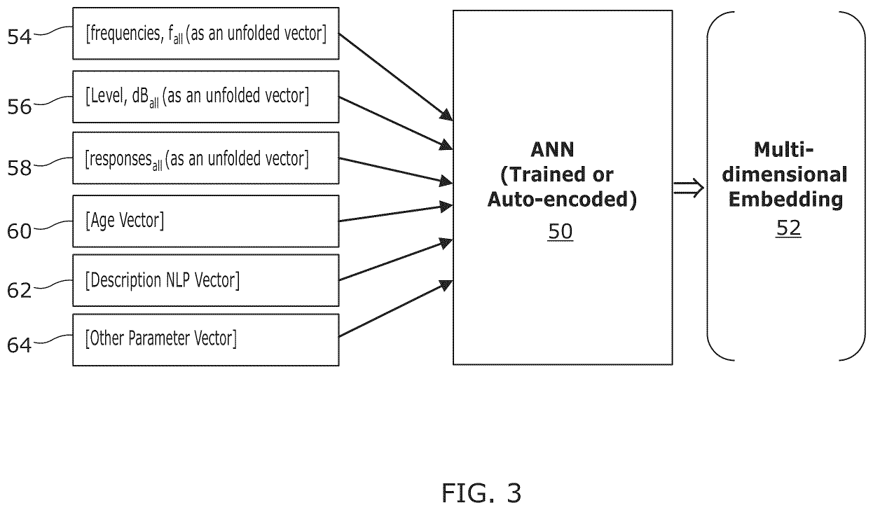

The combination of those frequency-level matrices provides a one-dimensional binary response in the form [x,x] where x is either zero or one depending upon a determined threshold of hearing. The same matrix approach applies to other selectable demographic parameters to yield a separate and distinct contributing embeddings of user-selectable length. This is shown in FIG. 3 in the context of the different vectorial inputs being applied through the ANN to produce a final embedding of, for example, 512 dimensions or some other user-defined length.

The eventual frequency-level pair output from the ANN would be a pro

$${f}_{1} {f}_{2} {f}_{3} {f}_{n}$$

form, following entry of data pertaining to parameters applied in construction of the network, resembling:

$$\{ Q ( {f}_{1} , 0 dB ) Q ( {f}_{1} , 5 dB ) … Q ( {f}_{1} , 25 dB ) … Q ( {f}_{1} , 40 dB ) \} = > Quality Value$$

where the Quality Value is a relative value, e.g., {0.5, 8.5, 0.27 . . . 0.3}.

The network is used to access the quality of a state-action pair, following the form of eqn. 1 which reflects the stepwise optimisation of the agent in RL. Once trained, the network predicts the next dB value given the recorded history of prior actions and the demographics.

FIG. 3 is a representation of an RL-based ANN 50 structured to provide acoustic loss evaluation according to an embodiment of the present invention. The ANN 50 provides, at its output, a multi-dimensional embedding 52. Preferred inputs to this neural network, most usually in the form of unfolded vectors or simply vectors, can include (but are not limited to):
i) all test frequencies (fₐₗₗ) 54,
ii) all associated levels (dBₐₗₗ) 56,
iii) all test responses (responsesₐₗₗ) 58,
iv) an age vector 60 identifying an age of the individual under test (whether for historical data for known audiograms required during training or otherwise for an actual test subject undergoing hearing loss assessment);
v) an optional NLP-generated description 62 that describes a sound environment exposure history related to an individual; and
vi) other optional parameters 64 selected as germane to hearing loss assessment, such as prevailing physiological conditions that degrade hearing or affect response.

Whilst not being an absolute, it has been found that RL agent training typically achieves convergence after training on ~100 k simulation steps. More or fewer may be required, as will be understood. Each simulation step is one complete audiometry “session” with a subject in the training set.

### C. Architecture

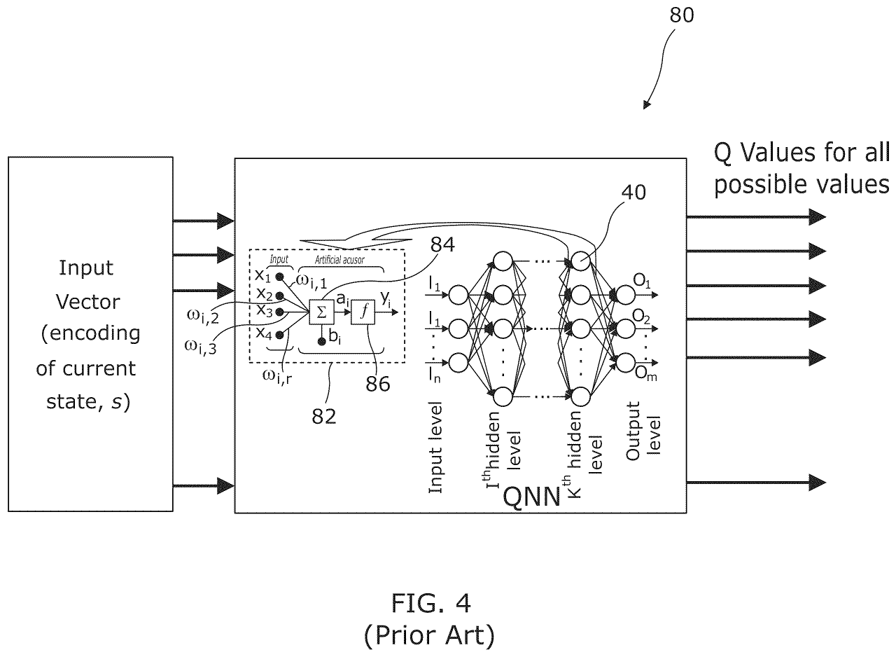

FIG. 4 is a representation of a typical artificial neural network 80 containing layers of neurons trainable and usable within the context of invention. Taking neuron 82 as an example, it can be seen in FIG. 4 (left side, boxed representation) that the neuron receives a plurality of weighted inputs w_{i,1}, w_{i,2}, w_{i,3}, w_{i,r} that are summed together in a summing function 84. The summing function, in fact, includes a secondary bias input bᵢ which is generally just a learned constant for each neuron in each layer. It is the weights wᵢ and the bias bᵢ that the processing intelligence estimates and then revises though a backpropagation process. An output aᵢ from the summing function 84 is subjected to a non-linear activation function f (reference number 86). The output of the neuron yᵢ is propagated to the next layer (containing many interconnected neurons) and so forth through the layers of the network, whether sparsely populated with fully interconnected neurons or fully populated).

By selection of a Deep Q-Network (“DON”), as described generally by Minh in the article “Human-Level Control Through Deep Reinforcement Learning” infra, as the basis for creating the RL agent for an application in audiometry, the present invention creates a neural network architecture which takes as input the state sₕₜ, extracts a learnable embedding vector of the state, and feeds that vector preferably to two fully-connected layers, although other layer types and sparse or pruned connections may be used. The final output of the network is a vector containing the predicted value of each action and, therefore, a probabilistic indication (or relative numeric values if the output is a quality function) of the likely next best test frequency-level pair to test at to identify or rapidly converge towards the likely lowest sound intensity (dB) level corresponding to the test subject's hearing threshold. In other words, the highest value is selected by system intelligence as the state frequency-level pair that is statistically expected, ultimately, to lead to an estimation of the audiogram in the least number of trials, with the selected state frequency-level pair generated and audible output.

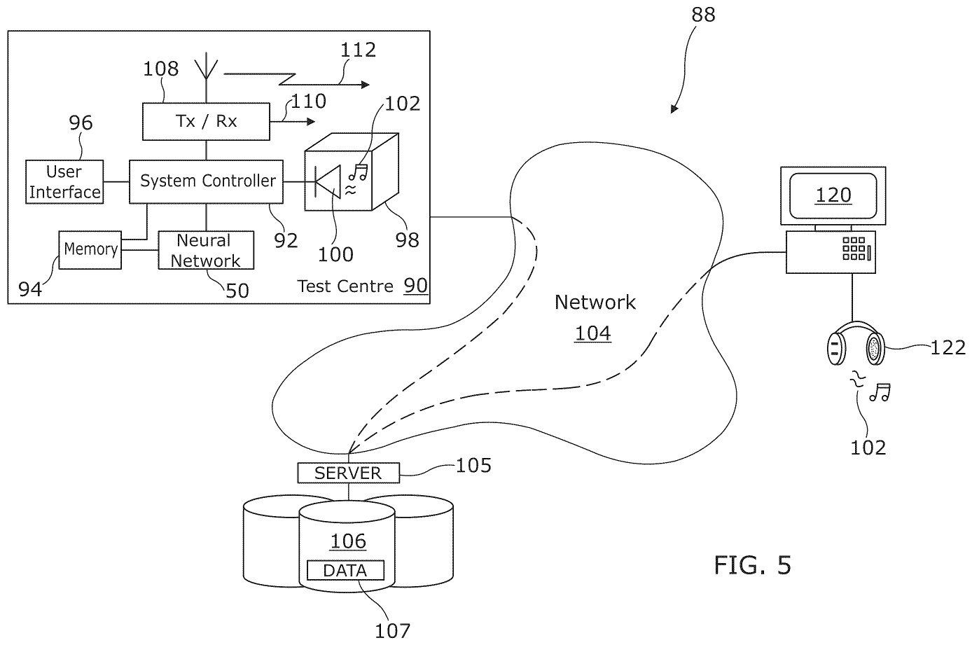

FIG. 5 shows an environment for an overall system 88 configured to support the audiology system of the preferred embodiments of the invention, as particularly exemplified in FIGS. 2 and 3.

At a specialist test centre 90, such as at a hospital or audiologist clinic, a system controller 92 maintains overall operational control of the hearing loss assessment process and system, as described herein. The system controller 92 is coupled to memory 94 which stores operational commands and locally generated data for use by the system. The memory may be independent of the agent memory (reference numeral 32 of FIG. 2), but it need not be since it serves as data storage and data access function. A trained neural network 50, set up according to the embodiments described herein, is responsive to the system controller 92 in that the system controller is able to oversee data flow presented to and results extracted from the neural network 50. The system 92 therefore includes a user interface 96 which typically includes a screen (relaying instructions to an end user/test subject) or graphic user interface “GUI” but at least includes some form of data entry mechanism to allow for responses to sound stimuli to be entered into—and therefore recorded by—the system. The user interface 96 also allows for entry of parameter data, such as the age 60 and description data 62 shown in FIG. 3 and outlined above. The user interface, which could also be an independent display screen, can also present data related to partial aspects of an entire audiogram.

The test centre also has a test enclosure 98, although this is optional. The test enclosure, if one exists, includes an audio output device 100, e.g., speaker or a headphone, arranged to generate the test tones 102 and intensity levels in response to instructions received from the system controller 92. The test centre may be a self-contained environment, but it may also be connected through a network 104 to a distributed system, such as a web application server 105 supporting a centralised ANN function and which server has access to data 107 storable in and retrievable from a database 106. The data can include historical audiograms as well as related or relevant information usable by the systems described herein. In a distributed configuration, certain data or functions, including the ANN 50, may therefore be located remote and offsite to the test centre 90, with access to distributed functions achieved via a transceiver chain 108 supporting bi-directional messaging. The transceiver may make use of wired 110 or wireless 112 connectivity to the network 104. Following hearing loss evaluation, control intelligence (typically at the server 106) can communicate, over the network, a resulting audiogram for a specific individual or, in a more limited sense, data related to compensation instructions, e.g., component tuning requirements or DSP processing instructions, required to mitigate identified hearing deficiencies.

Although the ANN-based audiology system can be based at a specialist site, it may also be supported on a local home environment, such as on a home computer 120. The computer 120, whether it this is a PC, smartphone or tablet, may support a full system architecture, but it may equally make use of the architecture of the distributed system 106 into which results are conveyed and analysed at a remote server with processing intelligence configured to oversee control of hearing evaluation. At the home environment [only which of which is shown in FIG. 5], the computer includes an audio output suitable for driving a tonal output from a headset 122 or another speaker set-up. The computer 120 can act directly as an input device for entering parameter data or responses to specific test tones at specific levels.

In a system in which the test is conducted in an uncalibrated environment, such as with off-the-shelf headphones, it is appreciated that headphone design is usually sculpted/tuned to provide a specific frequency response. If the headphone type is supplied by the user through an interface, then the specification—particularly relevant data 107—of the headphones being used can be retrieved from the database or otherwise entered (via the computer 120) or acquired by the server 105. The value in this headphone specification data is that the system can compensate for identified hearing deficiencies whilst also not doing so to an extent that compromises a soundstage specific to the specification of the headphones. If the headphone specification, for example, deliberately downplays certain higher frequencies, then boosting those higher frequencies in any compensatory instruction from the server for the headphones represent a potential error. Putting this differently, if the test is conducted through a set of uncalibrated headphones (including “earbuds” and the like) that actively and deliberately attenuate certain frequencies in an overall frequency response for the headphones [as designed by the manufacturer], then the provision of corrective instructions for identified hearing loss (in the user's audiogram) at those certain frequencies should take into account such manufacturer-specified design features. DSP, for example, can thus be used to boost frequencies to compensate for identified hearing loss deficiencies identified in a [supplied/determined] audiogram of the user, which audiogram (masked or precise) is resolved by the RL-based neural network approaches described herein.

In any forward pass, processing intelligence within the entirety of the system controls all aspects of the process, including the presentation or sending of the generated audiogram.

A preferred architecture for the neural network of embodiments of the present invention is a Deep Q-Network “DQN, such as described in the C. J. Watkins paper in Nature referred to above. DQN is a variant of a convolutional network, with DQN supporting a neural network approximator of the action “value” function. As explained above, this function gives the value of taking a certain action Aₜ given a state of the environment sₜ. The “value” of an action refers to its utility, or how much reward the agent is expecting to get in the future by performing that action. Actions with high expected utility have high value.

### D. Audio Loss Evaluation

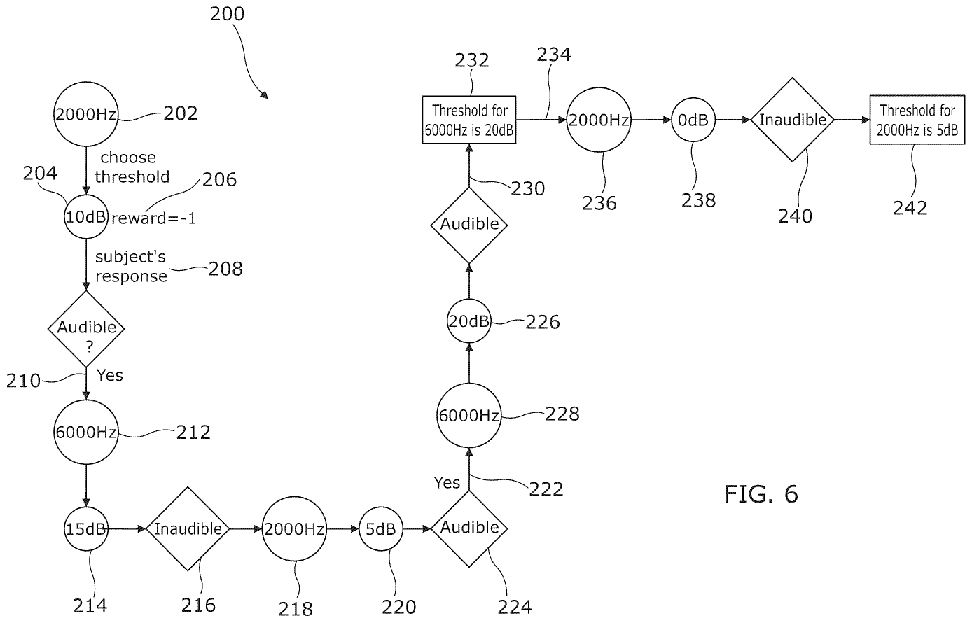

FIG. 6 shows an exemplary process 200 by which a threshold of hearing determination is assessed, albeit limited to the exemplary case of two frequencies within an overall audiogram. More particularly, FIG. 6 shows a decision process for an audiogram estimation on two exemplary frequencies, namely 2000 Hz and 6000 Hz.

Starting with a tone generated 202 at 2000 Hz, the agent (reference numeral 22 of FIG. 2) presents 204 a 10 dB tone to the subject and gets a negative reward consequential on the subject not being able audibly to resolve the tone at this level. The agent gives a negative reward 206 for presenting this 10 dB level [noting that the agent will continually generate negative rewards for each presented attempt]. The negative reward incentivizes the agent to minimize the number of interactions with the subject for estimating the overall audiogram. A negative reward is therefore associated with each newly presented level, particularly those which are unsuccessfully resolved for a specific test frequency. Conversely, if the subject responds 208, as in this exemplary case, that the tone is audible 210, the agent record this fact and moves on.

Given this initial detection at 10 dB at 2 kHz, the system controller selects a different frequency at a different level. This selection is influenced by historical audiograms and input parameter data such as demographics. For example, the control system selects 212 and presents 214 a 15 dB tone at 6000 Hz which, in this exemplary instance, the subject cannot 216 hear. The response to frequency interrogation and resolution—or lack thereof—by the audio nerve in response to localised sensitivity of the basilar membrane arising from physically close or indeed detection regions along the membrane, is accounted for in historical data to which the agent has access to on which it has been trained. Cooperation between the system controller 92 and agent 22 now function to select 220 a 5 dB level at 2 kHz (step 218), with this also resulting in (a) a negative reward being ascribed to the test by the agent, and (b) recordal of another response 222 from the subject that, under this exemplary enquiry, 2 kHz tone at this lower 5 dB level remains audible 224. At this point, the hearing loss/hearing threshold of the subject at 2 kHz remains undetermined.

The agent then selects and presents 226 a 20 dB sound level, again 228, at a tone of 6 kHz. In this exemplary instance, this tone is reported as audibly resolvable since this is the next dB HL increment. A hearing threshold for 6 kHz is 20 dB is therefore established 232. The agent then again selects 234 and presents 236 a 0 dB sound level at 2 kHz. In this instance, the subject is unable 240 to hear the tone at 0 dB (step 238), thereby establishing 242 that the threshold of hearing at 2 kHz is 5 dB. With the agent having found the hearing thresholds for both frequencies, the decision process terminates. The agent needed a total of 5 interactions with the subject and so, in this case, its total reward would be minus five (−5).

As soon as the hearing threshold is reached, the agent can and does abandon further testing at that test frequency. Similarly, the system is arranged to mask certain actions because they are already doomed to fail given prior responses. Particularly, the system operates to exclude testing at a level of 15 dB (and lower, i.e., a level of correction that is less than 15 dB) if the test subject has already provided a ‘cannot hear/negative’ response for the test frequency at a level of 20 dB [relative to perfect hearing]. This procedure is referred to as “action masking.” In other words, active masking is employed to the action value vector to rule out invalid thresholds, i.e., those thresholds which are outside of the subject's already established hearing range for the frequency already tested.

The demographic data together with the agent's memory is used by the agent to determine the next dB level, with the determination made based on the developed policy during training. The agent history, of course, continues to evolve and the stored to influence the next frequency-level state pairing, with the agent thus making this decision at blocks 204, 214, 220, 226 and 238 of FIG. 6.

### E. Masked Model

Masked modelling is tied to the concept of the approximation embodiment. The system's objective is to avoid testing at all frequencies, with the masked model predicting the hearing thresholds at any omitted test frequencies.

This is achieved by taking an audiogram from the dataset, randomly masking the thresholds for about ~40% of the frequencies, although this could be more or less, and presenting it to the agent and its active policy. The model is configured to predict the original audiogram before masking was applied. This is achieved by learning to minimize the error between its predictions for the missing threshold values and the actual values.

For the masked model, a transformer [see infra the article of A. Vaswani published in Advances In Neural Information Processing Systems, 30] provides a core basis for the model.

A Transformer-based architecture is able to incorporate information about all the known threshold values, across both ears, when predicting a masked threshold. This improvement was assessed relative to a recurrent neural network “RNN” based architecture.

The input to the masked model is the audiograms of the left and right ears of a subject, concatenated into a single vector and with some threshold values randomly masked. Each threshold value in this vector is considered as a token, with masked thresholds replaced with the “mask” token. For each token, an embedding is extracted. Additional embedding vectors are also extracted and contain information on the frequency and ear to which each threshold token belongs. This assumes the role of the positional embedding used in NLP applications. The masked model's output is a probability distribution over all possible thresholds, for each threshold in the input vector. Choosing the threshold with the highest probability is then made to be part of the final predicted audiogram.

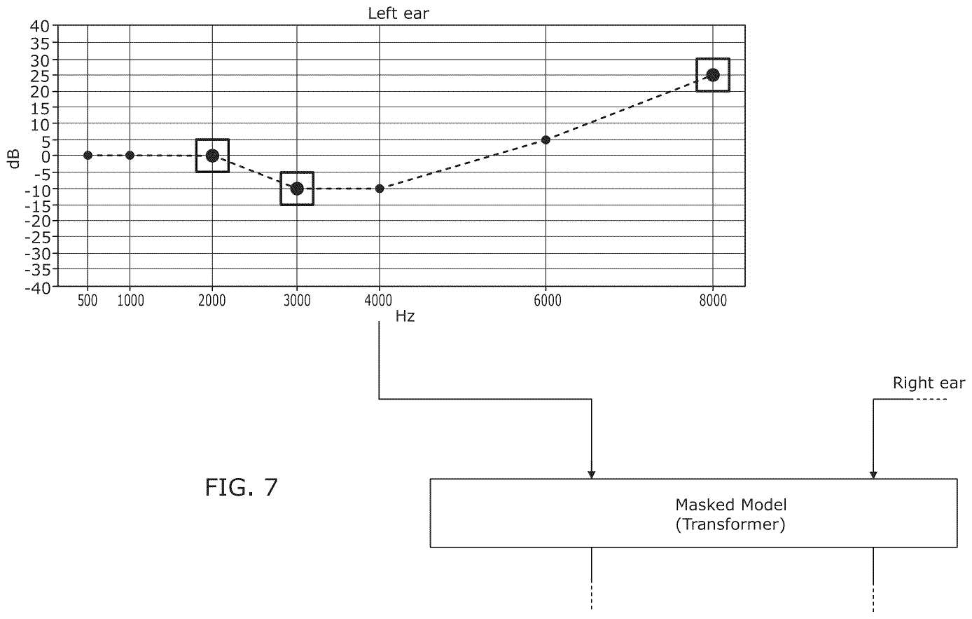

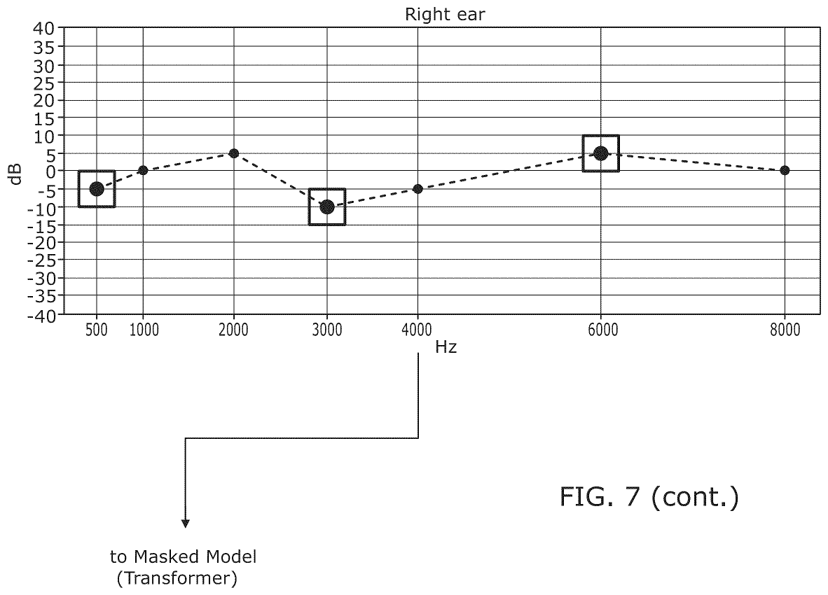

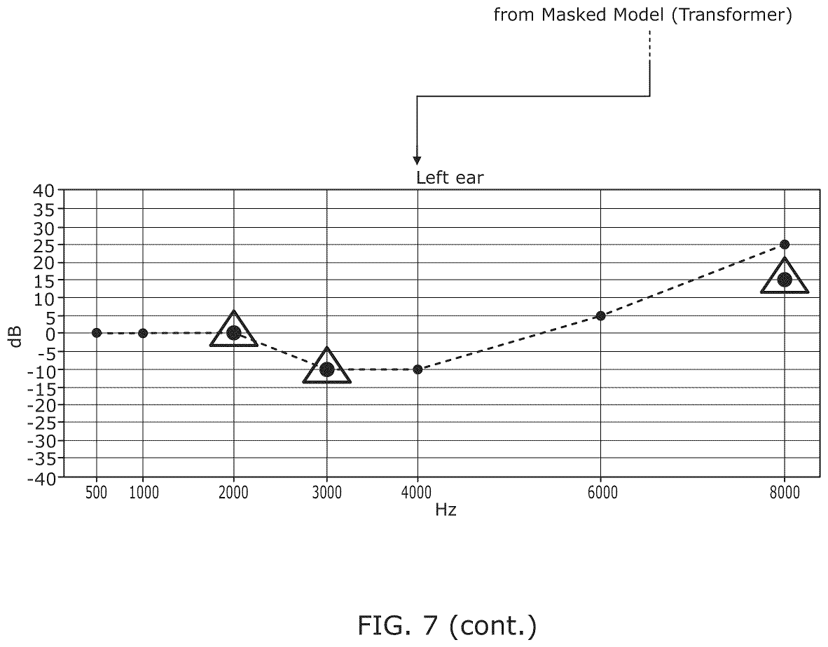

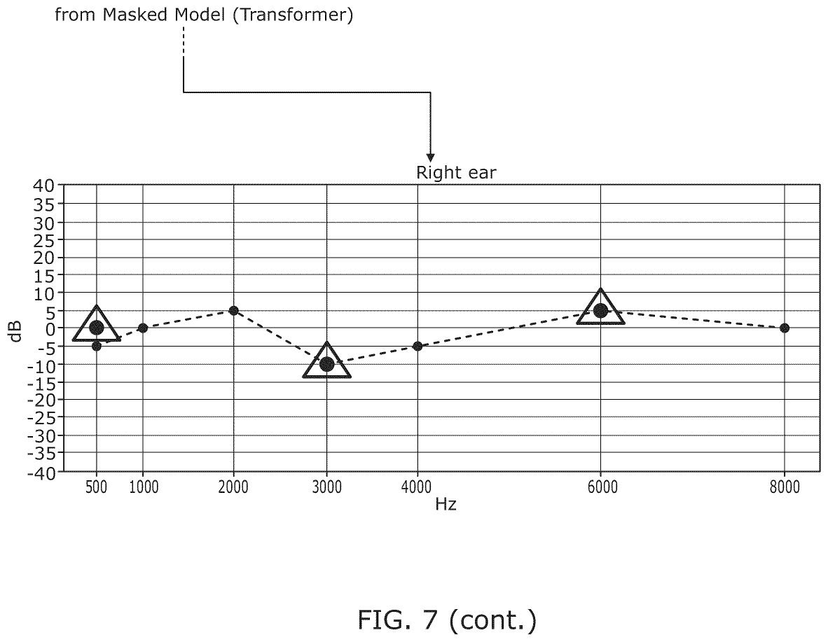

Reference is now made to FIG. 7 which shows the masking approach of this embodiment.

The model uses the known thresholds to predict the masked thresholds (marked at a centre point of a square) and arises by exploiting the relationship between thresholds at different frequencies as well as different ears. In FIG. 7, “filled-in” threshold values developed by the masked model are marked as the centre point of triangles.

The trained agent is used to measure the hearing thresholds, through interacting with the subject, for a subset of the available frequencies, and then the trained masked model is used to extrapolate the hearing thresholds for the other frequencies without requiring any further trials.

### Dataset

The NIOSH dataset contains audiograms, collected between 2000-2008, for male and female workers aged 18 and 65, in U.S. industries, who had higher occupational noise exposures than the general population. In this dataset, the workers were split into four age groups: 18-25, 26-35, 36-45, 46-55, 56-65. The frequencies tested were 500 Hz, 1 kHz, 2 kHz, 3 kHz, 4 kHz, 6 kHz, and 8 kHz. The hearing thresholds tested for each frequency were in the range-10 dB HL to +100 dB HL, with a granularity of 5 dB.

### F Experimental Results

Relative experimental results for “exact” and “approximate/masked” versions of the embodiments and approaches of the present invention are contrasted below relative to the methodologies employed by the automated Hughson-Westlake method (HW) and the Gaussian Process Classification method (GP). The comparison metric is the number of trials required to complete an audiogram.

Over the course of a randomly selected sample size of 500 subjects in each of the five age groups in the test set, each of the methods was run within a simulation environment (with known audiograms) and for each of the subjects. A mean was then taken for the required number of trials across all subjects. Table 2 below shows the mean number of trials required for audiogram estimation for each age group:

TABLE 2

| AGE | | | RL | Approx. RL |
|---|---|---|---|---|
| GROUP | HW | GP | (ours) | (ours) |
| 18-25 | 79 ± 8.1 | 56 ± 5.6 | 38.7 ± 5.5 | 22.5 ± 4.0 |
| 26-35 | 77.8 ± 8 | 59 ± 5.8 | 39.8 ± 6.8 | 22.7 ± 4.2 |
| 36-45 | 74 ± 7.3 | 63 ± 6.6 | 41.9 ± 7.5 | 27.7 ± 5.2 |
| 46-55 | 73.1 ± 7.5 | 68 ± 7.5 | 44.9 ± 8.3 | 28.6 ± 4.9 |
| 56-65 | 73.6 ± 7.6 | 71 ± 7.5 | 47.4 ± 7.7 | 32.2 ± 4.9 |

The RL-based approach of the present invention required 36% to 51% less trials than the HW method, depending on the age group, with the advantage being better for younger age groups. The masked version further reduced the number of trials by another 32% to 42% compared to the exact clinical version, again, depending on the age group.

By following the automated audiometry procedure as outlined by the paper infra “Online Machine Learning Audiometry” by Barbour et al, where each state pair lasts for a duration of two (2) seconds, the actual time required, on average, by each method to estimate an audiogram has been calculated. This is equal to the amount of time the subject must spend on the audiometry procedure itself, without including any preparatory phase. Table 3 below shows the approximate time (in seconds) that each method requires:

TABLE 3

| AGE | | | RL | Approx. RL |
|---|---|---|---|---|
| GROUP | HW | GP | (ours) | (ours) |
| 18-25 | 158 | 112 | 78 | 45 |
| 26-35 | 156 | 118 | 80 | 45 |
| 36-45 | 148 | 126 | 84 | 55 |
| 46-55 | 146 | 136 | 90 | 57 |
| 56-65 | 148 | 142 | 94 | 64 |

The exact (clinical) version of the RL-based method of a preferred embodiment reduces the time spent by the subject on the audiometry test to a range between about fifty-four (54) and eighty (80) seconds compared to the HW method, depending on the age group of the subject. The approximate/masked RL-based method of the invention was seen to reduce the time to one-hundred and thirteen (113) seconds compared to the HW method.

In modern digital devices, accessing at least some customisable settings through (a) a specific hearing aid's app or remote control thereof, or (b) adaptation of an output signal from an audio jack (or its digital equivalent) on a computer, in combination with ability to tweak/adjust dynamic settings through a related app, can improve an overall listening/hearing experience. The approximation/masked embodiment therefore lends itself to regular adaptation of audio equipment since the test procedures are now more readily available and rapid in execution. These effects allow for, especially, the masked version to be targeted at a consumer/home environment (reference numerals 120 and 122 of FIG. 5) in which improved audio experiences result from increased frequency differentiation, particularly as can be experienced within the context of gaming headsets and headphones.

The embodiments of the present invention therefore support adaptation of audio equipment, including hearing aids but more generally speaker or headphone output, to reflect and compensate for a personal audiogram determined quickly from either remote network-server access or from a locally installed app running on a home device.

Those skilled in the art will further appreciate that the various illustrative logical blocks, modules, circuits, methods, and algorithms described in connection with the examples disclosed herein may be implemented as electronic hardware, computer software, or combinations of both. To clearly illustrate this interchangeability of hardware and software, various illustrative components, blocks, modules, circuits, methods, and conceptual algorithms have been described above generally in terms of their functionality. Whether such functionality is implemented as a software emulation or in hardware or a combination depends upon the particular application and design constraints imposed on the overall system. Skilled artisans may implement the described functionality in varying ways for each particular application while remaining, either literally or equivalently, within the scope of the accompanying claims.

Unless specific arrangements are mutually exclusive with one another, the various embodiments described herein can be combined to enhance system functionality and/or to produce complementary functions or systems that support the effective identification of user-perceivable similarities and dissimilarities. Such combinations will be readily appreciated by the skilled addressee given the totality of the foregoing description. Likewise, aspects of the preferred embodiments may be implemented in standalone arrangements where more limited functional arrangements are appropriate. Indeed, it will be understood that unless features in the particular preferred embodiments are expressly identified as incompatible with one another or the surrounding context implies that they are mutually exclusive and not readily combinable in a complementary and/or supportive sense, the totality of this disclosure contemplates and envisions that specific features of those complementary embodiments can be selectively combined to provide one or more comprehensive, but slightly different, technical solutions. In terms of the suggested process flows implied or shown in the accompanying drawings, it may be that these can be varied in terms of the precise points of execution for steps within the process so long as the overall effect or re-ordering achieves the same objective end results or important intermediate results that allow advancement to the next logical step. The flow processes are therefore logical in nature rather than absolute.

As used in this application, the terms “component,” “module,” “system,” and the like are intended to refer to a computer-related entity, either hardware, firmware, a combination of hardware and software, software, or software in execution. For example, a component can be, but is not limited to being, a process running on a processor, a processor, an object, an executable, a thread of execution, a program, and/or a computer. By way of illustration, both an application running on a computing device and the computing device can be a component. One or more components can reside within a process and/or thread of execution and a component can be localized on one computer and/or distributed between two or more computers. In addition, these components can execute from various computer readable media having various data structures stored thereon. The components can communicate by way of local and/or remote processes such as in accordance with a signal having one or more data packets (e.g., data from one component interacting with another component in a local system, distributed system, and/or across a network such as the Internet with other systems by way of the signal).

It is understood that the specific order or hierarchy of steps in the processes disclosed herein is an example of exemplary approaches. Based upon design preferences, it is understood that the specific order or hierarchy of steps in the processes may be rearranged while remaining within the scope of the present disclosure. The accompanying method claims present elements of the various steps in sample order and are not meant to be limited to the specific order or hierarchy presented, unless a specific order is expressly described or is logically required.

Moreover, various aspects or features described herein can be implemented as a method, apparatus, or article of manufacture using standard programming and/or engineering techniques. The term “article of manufacture” as used herein is intended to encompass a computer program accessible from any computer-readable device or media. For example, computer-readable media can include but are not limited to magnetic storage devices (e.g., hard disk, floppy disk, magnetic strips, etc.), optical disks (e.g., compact disk (CD), digital versatile disk (DVD), etc.), smart cards, and flash memory devices (e.g., Erasable Programmable Read Only Memory (EPROM), card, stick, key drive, etc.). Additionally, various storage media described herein can represent one or more devices and/or other computer-readable media for storing information. The term “computer-readable medium” may include, without being limited to, optical, magnetic, electronic, electro-magnetic and various other tangible media capable of storing, containing, and/or carrying instruction(s) and/or data.

The above description has therefore been given by way of example only and that modification may be made within the scope of the present invention, such as the substitution of the QNN by a regression function based on a Δ change in an error signal with respect to time. The frequencies at which testing may occur are also open to change and a design choice. The testing range could be extended both to lower human audible frequencies (such as 100 Hz) and considerably higher audible frequencies (such as 16 kHz), as will be understood, and the step progression can be either linear or non-linear. Equally, the nature of the stimuli can be changed such that a single or very narrow frequency band can be replaced by compound stimuli, thereby extending the specific test environment/test set-up over multiple frequencies (whether adjacent/contiguous frequencies that produce a range for the test or pairs or triplets of frequencies separated by a frequency gap). Using broader frequency-based stimuli could therefore make use of phonemes or closely related words, e.g., shirt, skirt, flirt, hurt, as the basis for perceptual response, with the results then imported into an audiogram because the perceivability of phonetically similar words relates to audible resolution across frequency bands. This would result, potentially, in the use of word-level pairings rather than the exemplary case of frequency-level pairs at each test environment. Furthermore, using multiple frequencies has the potential to infer, from a recorded non-response, a minimum hearing threshold or an ability to move more radically to the next test stimuli.

In subject-testing in the forward pass, a non-response to a test stimulus can result from either the system timing out [for a response to be delivered by the test subject] or otherwise from the system providing, over a GUI, an active prompt [requiring a response from the test subject] directly asking whether the test subject has heard the frequency-level pairing?

### CLAUSES

The invention also contemplates the following embodiments listed in the numbered clauses below.
I. A method of modifying a frequency response of a speaker linked to a device and where
   the speaker has a manufacturer-defined frequency response, the method comprising: adapting processing parameters of the device, as overseen by control circuitry responsible for controlling soundwave generation from the speaker, in response to data identifying determined hearing thresholds at selected frequencies; and
   attenuating adaptation of the processing parameters when data identifying determined hearing thresholds at selected frequencies is determined to conflict with manufacturer-specified design performance of the frequency response of the speaker.
II The method of clause I, wherein the data identifying determined hearing thresholds at selected frequencies is based on a method of estimating and audiogram (and a related system) in which a neural network has been trained using a deep learning reinforcement framework set up with an agent and a neural network implementing a policy based on temporal difference learning, the audiometry system arranged to generate an audiogram in which a hearing threshold at a plurality of selectable test frequencies is identified, wherein the system is configured to: generate frequency-level pairings on a selective basis dictated by the policy, each frequency-level pair representing a current state of the environment; record, in memory, perception responses to those selectively generated frequency-level pairings presented from an audio output device; and identify a hearing threshold for those selectively generated frequency-level pairings to assemble the audiogram from perception responses for a plurality of different frequency-level pairings; wherein selection, by the agent based on its policy, of each frequency-level pairing takes into account the current state of the environment, sₜ, together with previous perception responses for different frequency-level pairings and defined parameter data pertaining to personal characteristics.
III. A method of estimating an audiogram, the method comprising:
   training a neural network using a deep learning reinforcement process to set an agent policy and in which the neural network is trained on a dataset of historical audiograms augmented by at least demographic data associated with each historical audiogram;
   recording a negative reward for each presented state pairing of a frequency-sound intensity level;
   following presentation of a first state pairing at a selected first test frequency and a selected first sound intensity level, selecting and moving to a second different state pair regardless of a determined interactive response;
   recording a first subject response with an association to the first state pair; and
   selecting and presenting a second state pair at a different second test frequency and different second sound intensity level, wherein selection of the second state pair is based on a probability outcome for the neural network taking into account at least the first subject response;
   oscillating, at least once, between the first and second frequencies whilst respectively changing respective sound levels presented thereat, thereby identifying a threshold level of hearing at each of the first and second frequencies; and
   generating a visual representation of the estimated audiogram based on reported positive responses to audio detection determined to be at a subject's threshold level of hearing at each frequency; and whereby consequently
   the agent policy is adapted to influence hearing loss evaluation strategy through selection of further state pairings based on responses to current and historical state environments and data related at least one of:
      (a) physiological characteristics of a test subject;
      (b) demographic data for a test subject; and
      (c) descriptive information describing a test subject.
IV. The method of estimating an audiogram according to clause III, further comprising:
   having an agent, as part of its action space, select a next frequency to be assessed; and
   having an agent select the level at the selected frequency.
V. The method of estimating an audiogram according to clause III or IV, further comprising:
   having an agent select the next frequency by following a predetermined sequence independent of the agent; and
   having an agent select the level at the selected frequency.
VI. The method of estimating an audiogram according to any of clauses III to V, further comprising:
   selecting and applying a mask configured to limit selection of test-frequency pairings.
VII. The method of estimating an audiogram according to clause VI, wherein the mask predicts hearing thresholds at selectively omitted test frequencies.

1. An audiometry system based on a neural network trained using a deep learning reinforcement framework set up with an agent and wherein the neural network implements a policy based on temporal difference learning, the audiometry system arranged to generate an audiogram in which a hearing threshold at a plurality of test frequencies is identified, wherein the system includes a system controller configured to:
control generation of selected word-level pairings within which are compound frequencies, the generation of the selected word-level pairing dictated by the policy, each word-level pair representing a current state of an environment;
effect recording, in memory, of perception responses to those selectively generated word-level pairings presented from an audio output device; and
identify a hearing threshold for word-level pairings to assemble the audiogram from perception responses for a plurality of different frequency-level pairings;
wherein selection, by the agent based on its policy, of each word-level pairing takes into account the current state of the environment, sₜ, together with previous perception responses associated with different word-level pairings and defined parameter data pertaining to personal characteristics.

2. The audiometry system of claim 1, further comprising at least one of:
presenting the audiogram on user interface; and
transmitter data from the audiogram over a network to a device located remotely to the audiometry system.

3. The audiometry system of claim 1, wherein the neural network is remote to audio equipment used to generate the word-level pairings as soundwaves, said audio audiometry system connected to the audio equipment over a network.

4. The audiometry system of claim 1, wherein inputs to the neural network during its training includes vectoral representations of at least some of:
i) all test frequencies (fₐₗₗ) within words of the word-level pairings;
ii) all associated levels (dBₐₗₗ);
iii) all test responses (responsesₐₗₗ);
iv) a test subject's age;
v) an NLP-generated description describing a sound environment exposure history related to a test subject; and
vi) parameter data related to physiological conditions that degrade hearing or affect perception response.

5. The audiometry system of claim 1, wherein the system is configured such that one of:
the agent, as part of its action space, is arranged to select a next word to be assessed;
selection of the next word follows a predetermined sequence independent of the agent; and in both configurations
the agent is arranged to determine the level.

6. The audiometry system of claim 1, wherein the neural network is trained by a Bellman equation process of the form
$$Q ( s , a ) ← Q ( s , a ) + λ ( R ( s , a ) + γ \{ {Q}_{{max}_{{a}^{′}}} ( {s}^{′} , {a}^{′} ) \} - Q ( s , a ) ) ,$$
where:
Q(s,a) is the current Q-value for taking action, a, in the current state, s,
λ is the learning rate which determines how much emphasis is placed on the new information to override/adapt the older Q-value,
R(s,a) is the immediate reward received after taking action a in state s,
γ is the discount factor, which weighs future rewards relative to immediate rewards, and
Qₘₐₓ_{ₐ},(s′,a′) is the maximum Q-value for the next state, s′, across all possible actions a′.

7. The audiometry system of claim 1, wherein selection of word-level pairings is subject to an applied mask configured to limit selection of test pairings.

8. An audio output system including control circuitry configured to be dynamically adaptable to support adjustment of a frequency response of the audio output system through adaptation of processing parameters overseen by the control circuitry, wherein the control circuitry is responsive to data relating to hearing thresholds determined through an on-line networked interaction of the audio output system with a reinforcement learning (RL)-based audiometry system based on a neural network trained using a deep learning reinforcement framework configured with an agent and wherein the neural network is arranged to implement a policy based on temporal difference learning, the RL-based audiometry system (88) arranged to generate an audiogram in which a hearing threshold at a plurality of selectable test frequencies is identified, wherein the system is configured to:
control generation of selected word-level pairings within which are compound frequencies, the generation of selected word-level pairing dictated by the policy, and wherein each word-level pair represents a current state of the environment;
effecting recording, in memory, of perception responses to those selectively generated word-level pairings presented from an audio output device; and
identify a hearing threshold for frequencies within the word-level pairings to assemble the audiogram from perception responses for a plurality of different word-level pairings;
wherein selection, by the agent based on its policy, of each word-level pairing takes into account the current state of the environment, sₜ, together with previous perception responses associated with different word-level pairings and defined parameter data pertaining to personal characteristics.

9. The audio output system of claim 8, wherein modification of the frequency response of the audio output system, to compensate for determined hearing loss thresholds values over a range of frequencies, is further modified to take into account manufacturer-specified design performance of the frequency response of the audio output system.

10. A hearing evaluation system based on a deep learning reinforcement framework configured with an agent and a neural network arranged to implement a policy and in which evaluation system intelligence is arranged to control overall operation of the evaluation system, wherein the neural network trained on a dataset of historical audiograms augmented by at least demographic data associated with each historical audiogram, and wherein a system intelligence is configured to:
record a negative reward for each presented state pairing of a plurality of word-level pairings with respective frequency-sound intensity components level; and
following presentation of a first state pairing with a first set of compound test frequencies with associated selected first sound intensity levels, to move to a second different state pairing regardless of an interactive response determined by the system intelligence;
record, as received by the system intelligence, a first subject response with an association to the first state pairing; and
select and present a second state pairing at a different second test set of compound frequencies and with different associated second sound intensity levels, wherein selection of the second state pairing is based on a probability outcome for the neural network taking into account at least the first subject response;
oscillate, at least once, between the first and second word-level pairings whilst respectively changing respective sound levels presented therein, thereby to identify a threshold level of hearing through audible resolution across frequency bands; and
generate a visual representation of an audiogram based on reported positive responses to audio detection determined to be at a subject's threshold level of hearing; and whereby consequently
the agent policy is adapted to influence hearing loss evaluation strategy through selection of further state pairings based on responses to current and historical state environments and data related at least one of:
(a) physiological characteristics of a test subject;
(b) demographic data for a test subject; and
(c) descriptive information describing a test subject.

11. The hearing evaluation system of claim 10, wherein the system is configured such that one of:
an agent, as part of its action space, is arranged to select a next word-level pairing to be assessed;
selection of the next word-level pairing follows a predetermined sequence independent of the agent; and in both configurations
the agent is arranged to determine the level.

12. The hearing evaluation system of claim 10, wherein audiogram estimation is formulated as a Markov Decision Process “MDP” optimised via reinforcement learning using a reward signal.

13. The hearing evaluation system of claim 10, wherein selection of word-level pairings is subject to an applied mask configured to limit selection of test pairings, and wherein the mask predicts hearing thresholds at selectively omitted test frequencies.

14. A method of evaluating a hearing level threshold, the method comprising:
training a neural network using a deep learning reinforcement framework set up with an agent and such that the neural network implements a policy based on temporal difference learning;
generating word-level pairings within which are compound frequencies, the generation of selected word-level pairing dictated by the neural network, each frequency-level pair representing a current state of an environment;
recording, in memory, perception responses to those selectively generated frequency-level pairings presented from audio output device; and
identifying a hearing threshold frequencies within the word-level pairings to assemble the audiogram from a plurality of perception response at a plurality of different word-level pairings;
wherein selection of each word-level pairing takes into account the current state of the environment, sₜ, together with previous perception responses associated with different word-level pairings and defined parameter data pertaining to personal characteristics and selection is made by the agent based on its policy; and the method further includes:
generating an audiogram in which a hearing threshold at a plurality of frequencies is identified; and
outputting the audiogram either on a user interface or transmitting data from the audiogram over a network to a device located remotely across the network.

15. The method of evaluating a hearing level threshold according to claim 14, wherein inputs to the neural network during its training includes vectoral representations of at least some of:
i) test frequencies (fₐₗₗ) within words of the word-level pairings;
ii) all associated levels (dBₐₗₗ);
iii) all test responses (responsesₐₗₗ);
iv) a test subject's age;
v) an NLP-generated description describing a sound environment exposure history related to a test subject; and
vi) parameter data related to physiological conditions that degrade hearing or affect perception response.

16. The method of evaluating a hearing level threshold according to claim 14, wherein one of:
the agent, as part of its action space, selects a next word-pairing to be assessed;
selecting the next word-pairing follows a predetermined sequence independent of the agent; and in both instances
the level at the selected word-pairing is determined by the agent.

17. The method of evaluating a hearing level threshold according to claim 14, wherein audiogram estimation is formulated as a Markov Decision Process “MDP” optimised via reinforcement learning using a reward signal.

18. The method of evaluating a hearing level threshold according to claim 14, wherein the neural network is trained by a Bellman equation satisfying a general form
$$Q ( s , a ) ← Q ( s , a ) + λ ( R ( s , a ) + γ \{ {Q}_{{max}_{{a}^{′}}} ( {s}^{′} , {a}^{′} ) \} - Q ( s , a ) ) ,$$
where:
Q(s,a) is the current Q-value for taking action, a, in the current state, s,
λ is the learning rate which determines how much emphasis is placed on the new information to override/adapt the older Q-value,
R(s,a) is the immediate reward received after taking action a in state s,
γ is the discount factor, which weighs future rewards relative to immediate rewards, and
Qₘₐₓ_{ₐ},(s′, a) is the maximum Q-value for the next state, s′, across all possible actions a′.

19. The method of evaluating a hearing level threshold according to claim 14, further comprising:
selecting and applying a mask configured to limit selection of test pairings.

20. The method of evaluating a hearing level threshold according to claim 19, wherein the mask predicts hearing thresholds at selectively omitted test frequencies.

21. The audiometry system of claim 1, wherein the compound frequencies are derived from a group selected from at last one of:
phonemes; and
closely related words.
